(19) United States
(12) Patent Application Publication
(10) Pub. No.: US 2024/0274426 A1
(43) Pub. Date: Aug. 15, 2024

(54) Automated Mass Spectrometry Sampling of Material Surfaces

(71) Applicant: Queen's University at Kingston, Kingston (CA)

(72) Inventors: Matthias Josef Hermann, Vellmar (DE); Haidy Metwally, Oakville (CA); Richard David Oleschuk, Kingston (CA)

(21) Appl. No.: 18/567,841

(22) PCT Filed: Jun. 10, 2022
(86) PCT No.: PCT/CA2022/050934

§ 371 Date: Dec. 7, 2023

Related U.S. Application Data
(60) Provisional application No. 63/209,216, filed on Jun. 10, 2021.

(51) Int. Cl. H01J 49/04; G01N 33/543

(52) U.S. Cl. CPC H01J 49/0413; G01N 33/543

(57) ABSTRACT

A controller for an open port interphase mass spectrometry (MS) probe automates analysis of samples, providing continuous, fast, and reproducible sampling. The sensed position of the probe above the sample surface is used by the controller in a feedback loop to set the probe at the proper position on the sample surface for sampling. One embodiment uses a conductance based sensor signal as input to the feedback loop to determine contact of the probe with the sample surface and to set the probe position according to a selected conductance value. The controller allows fast and automated sampling of uneven sample surfaces with minimal sample preparation while minimizing the risk of clogging the MS probe.

### FIELD

This invention relates to open port interface mass spectrometry for direct sampling of materials. More specifically, the invention provides apparatus and methods that enable automated mass spectrometry using a liquid microjunction-surface sampling probe. The apparatus and methods are particularly directed to automated sampling of uneven surfaces of biological and non-biological materials.

### BACKGROUND

Mass spectrometry (MS) imaging techniques are an important tool for the analysis of various materials, including biological samples such as tissue and bacterial colonies. The techniques combine the high sensitivity and specificity of mass spectrometry with spatial information by correlating recorded mass spectra to different locations across the sample. Heatmaps for different m/z values can be generated and used, for example, to distinguish between cancerous and benign tissue. Popular MS imaging techniques include direct analysis in real time (DART), desorption electrospray ionization (DESI), and liquid microjunction-surface sampling probe (LMJ-SSP). Regardless of the MS technique employed, such spectrometric imaging suffers from difficulty in achieving reproducible and quantifiable results for uneven surfaces due to varying distance between the sample surface and the MS probe.

### SUMMARY

According to one aspect of the invention there is provided an apparatus for automating movement of a mass spectrometry (MS) probe, comprising: a probe holder that is adapted to hold the MS probe and is moveable relative to a sample material surface along substantially planar x, y axes and along a z axis that is substantially orthogonal to the x, y axes; at least one z-axis sensor that outputs at least one sensor signal corresponding to a sensed z-axis position of a tip of the MS probe relative to the sample material surface, and; a controller that receives commands directing movement of the probe holder along at least the z axis, and receives the at least one sensor signal; wherein the controller uses the at least one sensor signal in a feedback loop to set the z axis position of the MS probe tip relative to the sample material surface.

According to another aspect of the invention there is provided a method for automating movement of an MS probe, comprising: using a probe holder to hold the MS probe, the probe holder being moveable relative to a sample material surface along substantially planar x, y axes and along a z axis that is substantially orthogonal to the x, y axes; providing at least one z-axis sensor that outputs at least one sensor signal corresponding to a sensed z-axis position of a tip of the MS probe relative to the sample material surface, and; providing a controller that receives commands directing movement of the probe holder along at least the z axis, and receives the at least one sensor signal; wherein the controller uses the at least one sensor signal in a feedback loop to set the z axis position of the MS probe tip relative to the sample material surface.

In some embodiments, the at least one sensor signal corresponds to an electrical parameter related to a z axis position of the MS probe tip relative to the sample material surface.

In some embodiments, the electrical parameter is conductance.

In some embodiments, the conductance value is greater than zero when the MS probe tip is in fluid communication with the sample material surface.

In some embodiments, the controller sets the z axis position of the MS probe tip according to a threshold value of the electrical parameter.

Some embodiments may comprise at least two z-axis sensors; wherein at least a first z-axis sensor outputs a first sensor signal corresponding to a z-axis distance of the MS probe tip above the sample material surface; wherein at least a second z-axis sensor outputs a second sensor signal corresponding to an electrical parameter related to a z-axis position of the MS probe tip upon fluid communication of the probe tip with the sample material surface.

In some embodiments, the controller receives commands directing movement of the probe holder along the x, y, and z axes, and receives the at least one sensor signal; wherein the controller controls movement of the MS probe to selected locations of the sample material according to the x and y axes commands, and the uses the at least one sensor signal in the feedback loop to set the z axis position of the probe tip relative to the sample material surface at each selected location.

In some embodiments, the sample material surface is uneven and the controller uses the at least one sensor signal in the feedback loop to set different z axis positions of the probe tip relative to the sample material surface at selected locations.

In some embodiments, the sample material comprises biological material.

In some embodiments, the sample material comprises non-biological material.

In some embodiments, the sample material surface is electrically conductive.

In some embodiments, the sample material surface is not electrically conductive.

In some embodiments, the MS probe is a liquid microjunction-surface sampling probe (LMJ-SSP).

According to another aspect of the invention there is provided non-transitory computer readable media for use with a processor, the computer readable media having stored thereon instructions that direct the processor to control an apparatus for automated mass spectrometry (MS) sampling, comprising: receiving input for selected parameters for controlling movement of an MS probe; generating and sending commands to the apparatus to control movement of the MS probe relative to a plurality of sampling locations across a sample surface; at each sampling location, moving the MS probe toward the sample surface in a z-direction until contact with the sample surface is achieved; recording z-height of the MS probe at each sampling location together with corresponding MS data; and generating an output comprising detected level of at least one analyte in the MS data at each sample location.

In one embodiment wherein the MS probe comprises a liquid microjunction-surface sampling probe (LMJ-SSP), the non-transitory computer readable media further comprises at each sampling location, moving the LMJ-SSP toward the sample surface in a z-direction until solvent contact with the sample surface is achieved.

In one embodiment the non-transitory computer readable media further comprises, at each sample location, moving the LMJ-SSP toward the sample in a z-direction until a feedback signal from a z-axis distance sensor is received; wherein the feedback indicates that solvent contact of the LMJ-SSP with the sample surface is achieved; wherein movement of the LMJ-SSP in the z-direction towards the sample is stopped.

In one embodiment, the z-axis distance sensor comprises a conductance sensor and the feedback signal indicating that solvent contact of the LMJ-SSP with the sample surface is achieved comprises a conductance value >0.

In one embodiment, the software records conductance values with respective times and relative position of the LMJ-SSP in x, y, and z-direction at each sampling location; and outputs mass spectra data assigned to respective spatial locations on the sample.

### BRIEF DESCRIPTION OF THE DRAWINGS

For a greater understanding of the invention, and to show more clearly how it may be carried into effect, embodiments will be described, by way of example, with reference to the accompanying drawings, wherein:

FIGS. 1A-1B, 1C, and 1D are diagrams of open port interface mass spectrometry probe and electrode configurations according to embodiments described herein.

FIG. 2A is a diagram showing an apparatus used to control movement of an LMJ-SSP, according to one embodiment.

FIG. 2B is a schematic diagram of a conductance sensor that may be used to implement a z-axis sensor, according to one embodiment.

FIG. 2C is a screen shot of a graphical user interface for computer software used for controlling automated sampling, according to one embodiment.

FIG. 2D shows inputs for computer software used for controlling automated sampling, according to one embodiment.

FIG. 3A is an LMJ-SSP-MS spectrum of a strawberry sample where characteristic peaks are labeled.

FIG. 3B is a photograph of the strawberry sample of FIG. 3A showing five sampling locations (a-e) along a line; the scale bar is 5 mm.

FIG. 3C is a total ion current (TIC) chromatogram for a range of m/z=150-500 where the peaks of each sampling location (FIG. 3B) are labelled.

FIG. 3D is an extracted ion chromatogram (XIC) for the same run as FIG. 3C for m/z=195 with a window of 1 Da showing where caffeine was detected.

FIGS. 3E-3I are mass spectra of each of the five sampling locations (a-e) where each is the average of 10 mass spectra in a time window of 8 s around the peaks of the TIC.

FIG. 4A is a plot showing relative conductance measured for an automated run of 10 sampling locations, using an embodiment of an apparatus as described herein.

FIG. 4B is a plot of z height of the probe relative to its starting height for the 10 sampling locations of FIG. 4A.

FIG. 5A is a photograph of a bovine tissue sample where the black rectangle shows the area spanning fatty and red tissue that was analyzed by LMJ-SSP-MS.

FIG. 5B is an enlarged image of the rectangular area in FIG. 5B that was analyzed, showing 20 sampling locations (four rows, each row including five locations indicated by “x”); each location was sampled for 8 s.

FIG. 5C shows four TIC chromatograms corresponding to the four sampling rows in FIG. 5B.

FIG. 5D is a heatmap of the analyzed area (20 locations) of FIG. 5B, wherein darker shading indicates greater heme concentration, and the graph at the top shows average values and standard deviations of each row.

FIG. 6A is a photograph of an area of an agar plate with four species of bacterial colonies.

FIG. 6B is a heatmap of the area shown in FIG. 6A that was sampled at 144 equally spaced locations arranged in a 12×12 grid, showing the spatial distribution of prodigiosin produced by the bacteria in the lower left corner of the area.

### DETAILED DESCRIPTION OF EMBODIMENTS

In open port interface mass spectrometry imaging techniques for the analysis of materials, including, e.g., biological samples such as tissue and bacterial colonies, a consistent distance between the mass spectrometry probe and the material surface is crucial to obtaining reproducible and quantifiable results, since the probe must be maintained in fluid communication with the surface so that the liquid droplet (i.e., a desorption solvent) at the probe tip contacts the material surface, allowing analyte(s) to be collected and transferred to the mass spectrometer. Maintaining a constant probe to sample distance may be easily achieved for flat and level samples by moving the probe to a consistent z height (i.e., z-axis) above the sample surface. Analysis of uneven sample surfaces (e.g., tissue samples, materials that have not been thinly sliced, agar plates with protruding bacterial colonies, etc.) can be tedious and problematic to analyze because the distance between the probe and the sample needs to be manually adjusted, often for each measurement.

Described herein is a feedback control system that may be used to automate sampling of uneven surfaces for liquid microjunction-surface sampling probe mass spectrometry (LMJ-SSP-MS) imaging.

As used herein, the term “uneven surface” refers to a sample material surface that may not be planar; that is, a surface that exhibits one or more peaks and valleys such that the difference in height between a peak and a valley would be sufficient to negatively affect the ability of an LMJ-SSP to be in proper fluid communication with the sample material surface if the LMJ-SSP were deployed at a substantially constant Z-axis height at sample locations during sampling. The uneven surface may result from sample material preparation, or the uneven surface may be an inherent property of the sample material. The term “uneven surface” also refers to a sample material surface that is substantially planar, but is sloped or inclined with respect to the z-axis.

As used herein, the term “z-axis” refers to an axis travelled by a probe that is substantially perpendicular with respect to a sample material surface. The term “substantially” is used because a sample material surface may be an uneven surface and accordingly the z-axis may be only approximately perpendicular to a given location on the surface.

Embodiments are based, at least in part, on sensing z-axis distance of the LMJ-SSP tip above the sample surface, and using the sensed distance in a feedback loop to automatically control the z-axis height of the probe tip to achieve the correct distance for sampling. Various technologies may be employed for sensing the z-axis distance of the probe above the sample surface. For example, depending on physical characteristics of the sample, the sensor may be a proximity sensor based on light (visible, infra-red, etc.), ultrasound, etc., or an electrical sensor that measures change in an electrical parameter with distance from the sample or contact with the sample, or a combination thereof. Embodiments may include controlling the x- and y-axis positions of the LMJ-SSP according to predetermined (e.g., programmed) positions, and controlling the z-axis position of the LMJ-SSP above the sample surface according to the sensed distance of the LMJ-SSP tip above the sample surface.

In one embodiment, a feedback control system may be implemented based on measuring conductance between an LMJ-SSP and a sample surface. For example, a conductance value may be >0 when the LMJ-SSP tip touches the sample. According to one embodiment based on measuring conductance, when the probe tip, which may be of a metal or silica material, liquid (i.e., MS desorption solvent mixture) droplet at the probe tip, and sample surface are in contact an electrical circuit is completed and conductance may be measured. Here, the probe tip may not physically contact the sample; rather, fluid communication between the probe tip and the sample surface is established by the liquid droplet.

In another embodiment a feedback control system may be implemented based on a combination of sensors. For example, the combination of sensors may include a proximity sensor (e.g., based on light) and a conductance sensor. The proximity sensor may allow the probe tip to be moved rapidly (i.e., at a first rate of travel) in the z-axis toward the sample surface until a threshold minimum distance from the sample surface is detected. The probe tip may then be moved slowly (i.e., at a second rate of travel) in the z-axis toward the sample surface until proper contact (e.g., fluid communication) with the sample surface is detected using the conductance sensor. Such an embodiment may provide more rapid automated sensing of an uneven sample material surface at multiple locations by enabling two (or more) rates of travel of the probe in the z-axis, which reduces the time interval between measurements.

Embodiments described herein provide various degrees of automation. For example, in one embodiment, control of only the z-axis height may be automated, with manual x-axis and y-axis positioning of the LMJ-SSP across a sample. In another embodiment, control of all three axes (z, x, y) may be automated. An LMJ-SSP mount may be based on a platform designed for such three-axis movement, or on a robotic arm. The latter may advantageously allow rotational movement of the LMJ-SSP, and thus enable MS imaging of a three-dimensional sample. For example, embodiments may be implemented as a portable/mobile MS machines adapted for use in environments other than laboratories, such as in hospitals, clinics, airports, farms, environmental assessments, or other facilities.

Embodiments described herein enable automated sampling of uneven surfaces of biological materials (e.g., such as but not limited to tissue samples, materials that have not been thinly sliced, agar plates with protruding bacterial colonies, bacterial scaffolds, biofilms, etc.), as well as non-biological materials (e.g., such as but not limited to equipment surfaces (such as in hospital surfaces, including bed rails, operating room surfaces, etc.), HVAC filters and ducts, seating, cotton balls, surgical sponges, etc.).

Another aspect of the invention relates to computer software that may be used with a feedback control system to automate sampling of uneven surfaces for liquid microjunction-surface sampling probe mass spectrometry (LMJ-SSP-MS) imaging. Embodiments may be implemented in computer code executable by a computing device or network of computing devices (e.g., one or more laptop computer, tablet, personal computer (“PC”), desk top computer, server, etc.), referred to generally herein as a computer. The computer may include an input device, a processor (e.g., a central processing unit (CPU)), memory, a display, and an interface device. The input device may include a keyboard, a mouse, a trackball, a touch sensitive surface or screen, or a similar device. The display may include a computer screen, television screen, display screen, terminal device, a touch sensitive display surface or screen, and/or a hardcopy producing output device such as a printer or plotter.

Such computer may include a non-transitory computer-readable storage medium, i.e., storage hardware, non-transitory storage device, or non-transitory computer system memory, etc., having stored thereon the computer code, i.e., computer-executable instructions, software program, an application (“app”), etc. that may be accessed by the processor. Accessing the computer-readable medium may include the processor retrieving and/or executing the computer-executable instructions encoded on the medium, which may include the processor running the app on the computer. The non-transitory computer-readable medium may include, but is not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like. Executing the stored instructions may enable the computer to carry out processing steps in accordance with embodiments described herein, for example, controlling x-, y-, and z-axis movement and positioning of an LMJ-SSP, collecting and processing of data obtained from one or more sensors including a z-axis distance sensor, collecting and processing of one or more measured variables, outputting results, data, etc., and may include prompting a user for input. Features of software embodiments are described in detail in Examples 1, 2, and 3 below.

The invention will be further described by way of the following non-limiting examples.

### Example 1

This example describes experiments demonstrating implementation of various embodiments.

Mass Spectrometry. Mass spectra were acquired on an API 3200 and an API 4500 triple quadrupole mass spectrometer (Sciex, Concord, ON). The mass spectrometer was equipped with a prototype open port interface LMJ-SSP developed in-house for sample introduction.

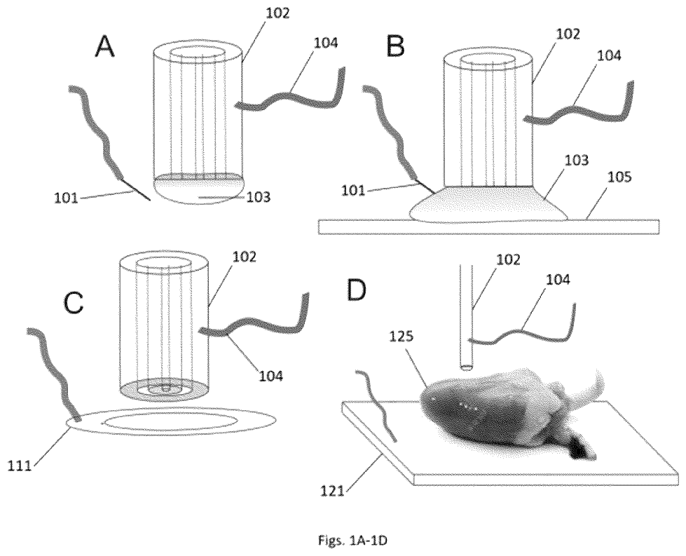

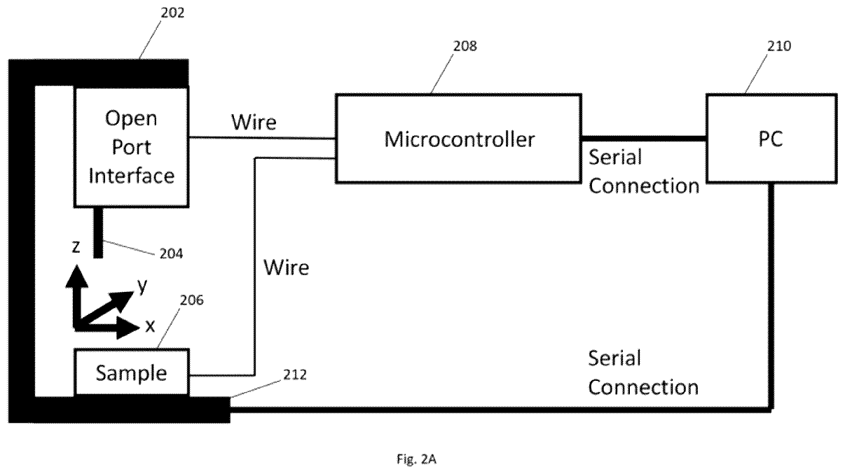

Various LMJ-SSP/electrode configurations may be employed to enable samples with different conductivities to be examined, three examples of which are shown in FIGS. 1A-1B, 1C, and 1D. Referring to FIG. 1A, an electrode 101 is suspended adjacent to and independent of the probe 102 which has a droplet of MS solvent mixture 103 at its tip and an electrical connection to wire 104. Many organic and aqueous solvents may be used either as pure solvent or in mixtures such as, but not limited to, methanol, acetonitrile, dichloromethane, methanol:water mixture with or without acid (e.g., formic acid, acetic acid), acetonitrile:water mixture. In FIG. 1B the droplet 103 spreads and contacts the electrode 101 when it touches the sample surface 105, completing the electrical circuit. This configuration enables non conductive materials to be used with conductivity touch positioning of the probe tip via the droplet. Alternatively, in the embodiment of FIG. 1C a ring electrode 111 may be used so that sensing is achieved no matter the direction of droplet wetting on a sample surface. FIG. 1D shows an embodiment wherein the electrode 121 is configured for conductive samples and the circuit is completed when the probe makes contact with the sample 125 (e.g., biological tissue or organ, agar plate, etc.). In this embodiment the electrode wire is connected to a conductive substrate 121 which may be a metallic plate (e.g., aluminum foil), indium tin oxide (ITO) coated glass, etc.

In this example the LMJ-SSP had two concentric tubes that were substantially coterminous at their distal ends (i.e., closest to the sample). The outer tube was made of stainless steel (OD=1.2 mm, ID=1 mm) and had a structure that accommodated the smaller inner tube made of polymer (polyetheretherketone, PEEK) (OD=0.8 mm, ID=0.25 mm). A solvent mixture was delivered into the space between the outer and inner tubes from a proximal end with a consistent flowrate. When the solvent mixture (methanol:water:formic acid, 50:49.9:0.1 vol. %) reached the distal end of both tubes a liquid dome was formed at the terminus of the tubes due to the surface tension of the liquid. The liquid forming the dome was continually drawn into the inner tube which was connected at the proximal end to the electrospray ionization (ESI) source of the mass spectrometer due to the Venturi effect.

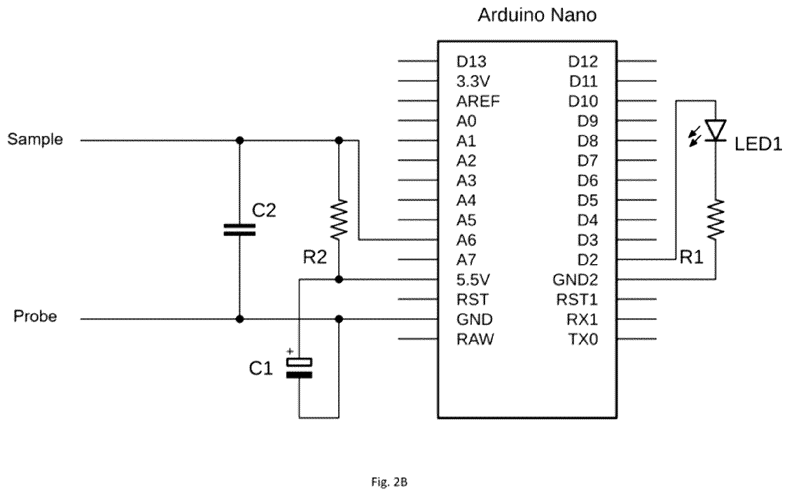

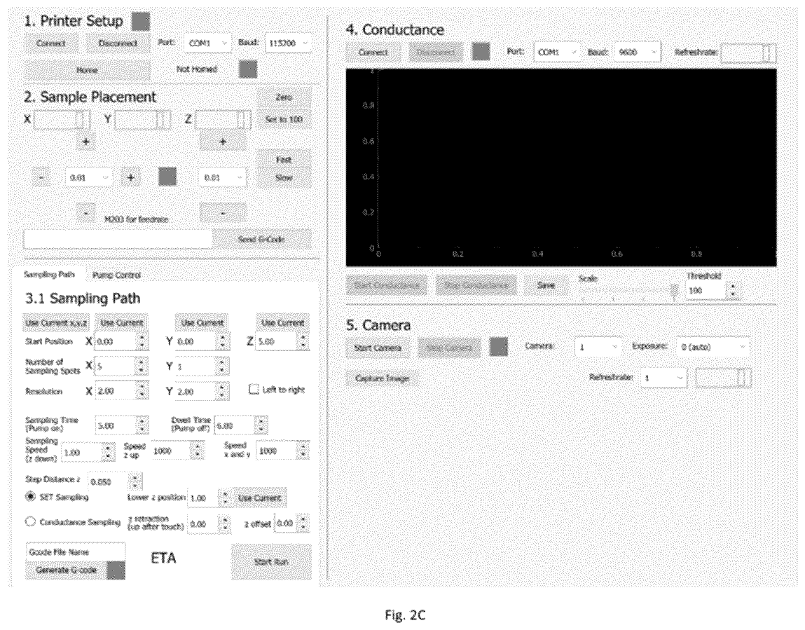

Apparatus. Apparatus used to control movement of the LMJ-SSP, according to one embodiment, will now be described with reference to FIG. 2A. The printer head 202 of a 3D printer (Prusa i3-MK3) was modified and used to hold and move the LMJ-SSP 204 relative to a sample 206 placed on the printing bed 212. A 3D printer was used as it presented an apparatus that could conveniently be adapted to hold the probe and control positioning of the probe relative to the sample using a computer numerical control (CNC) programming language such as G-code. The apparatus may be based on other machines that similarly enable or are adaptable for programming movement of a probe in multiple (e.g., three) axes. It is noted that such apparatus may be based on designs wherein the probe holder (e.g., printer head) moves in all axes (x,y,z) while the bed is stationary, or in other designs wherein the probe holder moves in one or two axes while the bed moves in the remaining axis or axes, or in other designs wherein the bed moves in all three axes. The apparatus may of course be custom-designed. Modifications included attaching a custom LMJ-SSP holder to the printer head to hold the probe in the correct position relative to the sample. A controller was implemented using a computer 210 running G-code and was used to control movement of the printer head 202, and hence the LMJ-SSP 204. A microcontroller 208 was used to interface a z-axis distance sensor with the computer. An embodiment of a z-axis sensor is described in Example 2.

G-code files were generated and sent to the 3D printer using a Python™ script (Python Software Foundation, version 3.7.0). Automation included movement along the z-axis (i.e., distance above the sample surface) according to a feedback loop, and movement along the x-axis and/or y-axis to control a sampling path, i.e., stepping of the probe across locations on the sample. The z-axis distance sensor was implemented with a conductance sensor, e.g., the embodiment shown in FIG. 2B. The microcontroller 208 measured the electrical conductance between the outer steel tube of the LMJ-SSP and the sample surface and implemented the feedback loop using the conductance value as an input to control z-axis movement. In general, moisture content of the sample provides sufficient conductance to trigger the feedback loop once the LMJ-SSP is in contact with the sample. When the LMJ-SSP touches the sample surface the conductance value is >0. The G-code script was designed to move the probe toward the sample while measuring the conductance value. Once a predetermined conductance threshold value was reached, movement of the probe towards the sample was stopped. The probe was then at a z-axis position above the sample surface that provided fluid (i.e., liquid) communication between the probe and the sample.

In one embodiment, G-codes that controlled movement of the open port interface relative to the sample are based on several inputs shown in Table 1. Whereas the example relates to sampling a rectangular grid, it will be appreciated that codes can easily be generated to control sampling over any shape of sample area. The generated G-code of the sampling path was exported and sent to the 3D printer which performed the corresponding sampling movement. In one embodiment, two modes were implemented for automated sampling by the 3D-printer—“constant z height mode” and “variable z height mode”. In both modes, a rectangular area of the sample surface was sampled line by line. In constant z height mode, the probe was lowered to the same z height at each sampling location to touch the sample. Therefore, this mode requires the sample surface to be even (i.e., flat) and leveled across the whole area that is sampled. This mode may be used, e.g., for sliced tissue sections or droplet arrays on patterned surfaces. In variable z height mode the probe was moved above the sampling location and was then lowered towards the sample until the conductance feedback loop is triggered. This mode allows sampling of uneven, and unlevel samples. In this mode, the high cost and time-consuming process of precise sectioning of samples such as tissue to the thickness of tens of micrometers is avoided.

TABLE 1
G-code input values used to create a rectangular sampling grid.

| Input | Description |
|---|---|
| Start x | x position of first sampling location |
| Start y | y position of first sampling location |
| Start z | z position above first sampling location where LMJ-SSP moves before |
| | approaching first sampling location |
| Lower z (non- | z position (height) of all sampling locations. This value is below Start z |
| conductance mode) | |
| Upper z | z position (height) the LMJ-SSP moves to after sampling at lower z |
| Sampling time | Time in seconds the LMJ-SSP resides in the lower z position (in contact with |
| | the sample) at each sampling location |
| Pause time | Time in seconds the LMJ-SSP resides in the upper z position (above the |
| | previous sampling location) to allow the sample to reach the detector before |
| | approaching the next sampling location. |
| Resolution x | Distance between each sampling location in x-direction in mm |
| Resolution y | Distance between each sampling location in y-direction in mm |
| Locations x | Number of sampling locations in x-direction |
| Locations y | Number of sampling locations in y-direction |
| x retraction | Distance in mm the LMJ-SSP moves up immediately after the conductance |
| (conductance mode) | loop is triggered. |

Optional features of z-axis control may be implemented in the G-codes to enhance performance and/or fine-tune control of the z height of the probe. Variations of the sampling regime may be implemented according to one or more of the type of material being sampled, humidity, temperature, salinity, thickness, roughness, unevenness, and the desorption solvent used. Also, the amount of time that the probe is in contact with the sample material may be optimized for one or more of a given material, analyte, thickness, solvent/solution/sample viscosity, sampling conditions, etc. The conductance threshold may be set according to the conductivity of the solvent and/or the sample/medium. Since the conductivity of a sample depends on its moisture content, electrolyte concentration, etc., different samples may have different conductivities and this may require optimizing the sensitivity of the conductance sensing circuit, e.g., adjusting a resistor value in the circuit (e.g., the value of R2 in the embodiment of FIG. 2B) or adjusting the required threshold value in the software.

For example, in some cases the probe may be raised slightly from the sample surface after contact is detected to minimize potential clogging. For example, a retraction value may be added to the code to move the probe away form the sample surface by a selected z-distance distance immediately after contact between the probe and the and the sample surface is detected (e.g., by a conductance value resulting from fluid communication with the sample surface). Retraction of the probe may be used to increase the size of the liquid junction between the probe and the sample surface for increased extraction efficiency, while reducing lateral spreading of solvent which would reduce lateral resolution of sampling.

As another example, one or more z-position values from one or more previous sampling locations may be stored and used to determine or approximate a z-position for a next sampling location. In one embodiment the controller may store one or more previous z-position values and estimate the next z-position according to a selected amount of the previous one or more values, e.g., 80%, or 90%, or 95% of the one or more previous values. This way the controller may implement variable-speed z-axis control of the probe by rapidly advancing the probe to the estimated z-position and then slowly advancing the probe until proper fluid communication is established with the material surface, as confirmed by a conductance sensor. Such an embodiment significantly reduces the overall sampling time by reducing time between automated measurements.

Data Analysis. MS data were collected on a computer running Analyst (1.6.3 Sciex, Concord, ON) and were exported in *wiff format. MS Convert was used to convert *wiff files to *mzml files. Mass spectra that were recorded during contact with the sample were extracted from *mzml files and saved and processed as *csv files. Data were plotted using resources available from the Plotly Python Open Source Graphing Library.

### Results

Caffeine Spiked Strawberry Samples. Fresh strawberry slices were used to demonstrate direct analysis of biological samples by manually positioning the LMJ-SSP. To spatially differentiate between different areas of the sample the right half of a strawberry slice was spiked with 1 μL of an aqueous solution of caffeine (1 mg/mL).

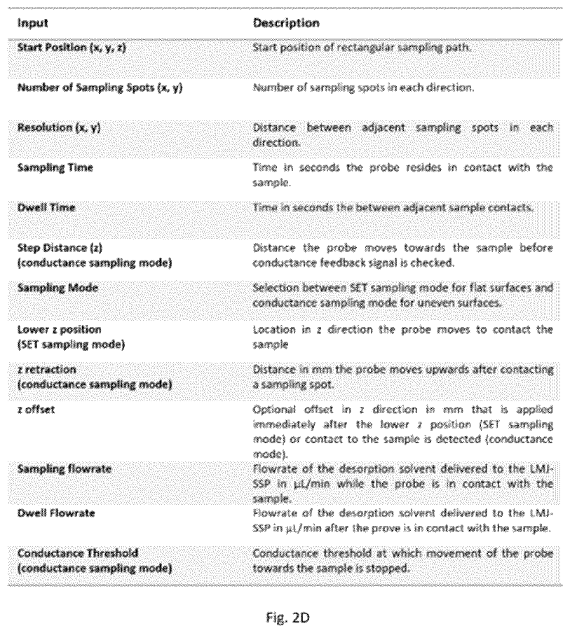

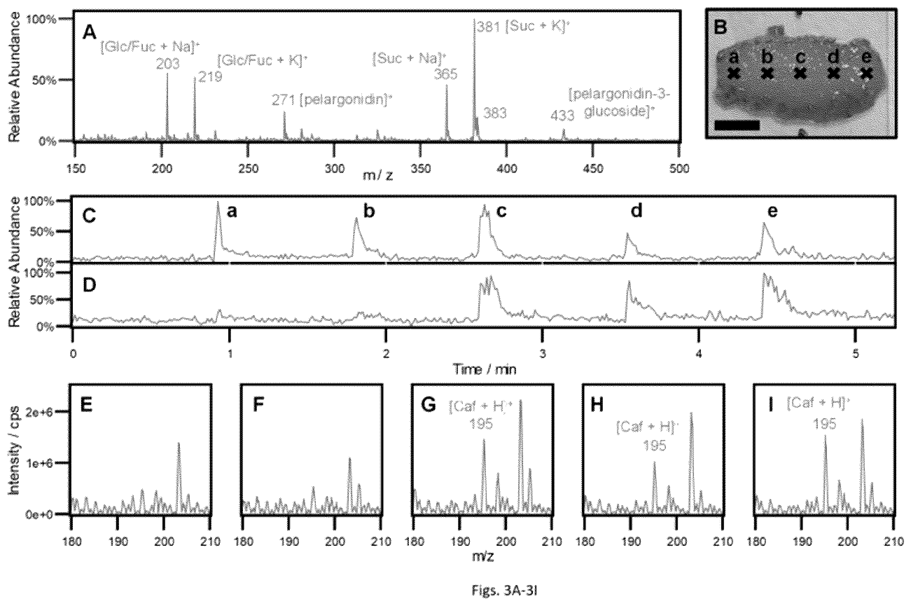

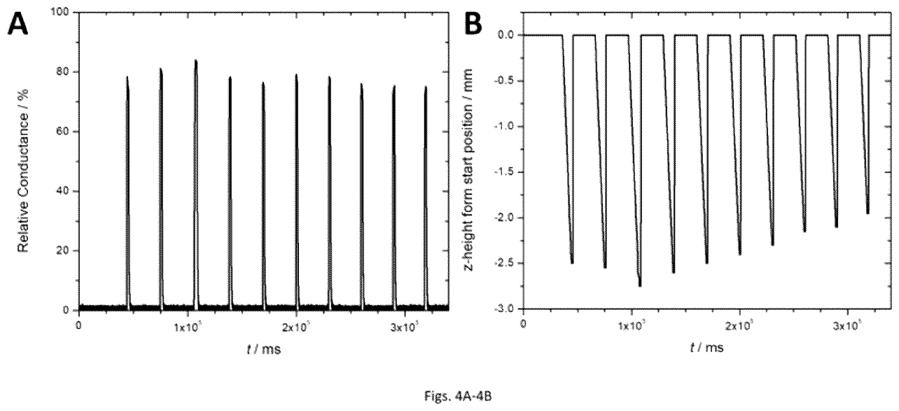

The LMJ-SSP was mounted on a modified 3D printer head as described above. The strawberry slices were sampled by manually controlling movement of stepper motors of the 3D printer head in x-, y-, and z-directions to change the relative position of the LMJ-SSP relative to the sample placed on the printing bed. FIG. 3A shows the mass spectrum of a strawberry slice that was directly sampled by LMJ-SSP-MS. The sodium (m/z=203) and potassium (m/z=219) adducts of glucose and fructose are easily identifiable. Furthermore, the potassium adduct of saccharose can be seen at m/z=381 and 383. Pelargonidin-3-glucoside (m/z=433) and pelargonidin (m/z=271) were detectable with lower intensities. The strawberry slice was sampled at five evenly spaced locations across the sample. The LMJ-SSP was aligned above the desired sampling location manually by moving the modified 3D printer head, and then slowly moved towards the sample until it was wetted by the solvent from the LMJ-SSP. After 6 s the LMJ-SSP probe was raised and aligned above the next sampling location and the process was repeated. FIG. 3B shows the five sampling locations, labelled a to e. FIG. 3C shows the associated total ion chromatogram (TIC) for m/z=150-500. The peaks for the five sampling locations a to e are identifiable and labeled according to their positions on the strawberry slice (FIG. 3B).

The distribution of caffeine on the sample can be reconstructed by the XIC in FIG. 3D (m/z=195, width=1 Da). While the first two sampling locations (a and b) do not show a peak, the other three (c, d, e) show the presence of caffeine. FIGS. 3E-3I show mass spectra of each sampling point (a to e). These spectra are average spectra of a time window of 8 s around the TIC peaks (10 spectra with a scan time of 0.8 s each). All mass spectra show the characteristic peak for the sodium adduct of glucose/fructose at m/z=203. In FIGS. 3G-3I the caffeine peak at m/z=195 can also be seen.

The procedure of moving the LMJ-SSP manually was slow and tedious because each sampling location needed to be slowly approached from above until the probe was in contact with the sample. Moving the probe too fast or without full attention towards the sample can result in the probe being pushed into the sample, leading to flow interruption and possible clogging of the probe. In addition, the time the probe is in contact with the sample is difficult to control when the movement is manually controlled.

Verification of Conductance Measurements for Z-axis Control. Preliminary data were obtained to verify that conductance measurements could be reliably obtained and used to control proper z-axis positioning of the LMJ-SSP using the embodiment described above. FIG. 4A is a plot of exemplary relative conductance measurements taken at 10 consecutive sampling locations across an uneven surface of a porcine tissue sample. It can be seen that the measurements are highly consistent. FIG. 4B is a plot of the z height of the probe relative to its starting height for the 10 sampling locations of FIG. 4A. It can be seen that there is variation in the z-height, which results from the uneven surface of the sample material.

Heme Heatmap of Bovine Tissue Sample. An automated feedback system based on conductance measurements as described above was used in variable z height mode to create a heatmap of heme distribution on a bovine tissue sample with minimal sample preparation, including rinsing the sample for about 10 s with water and cutting it to an uneven thickness of about 10 mm±3 mm. The LMJ-SSP was moved automatically to 20 locations arranged in a 5×4 grid at the interface of fatty and red tissue to obtain a heatmap of the spatial distribution of heme.

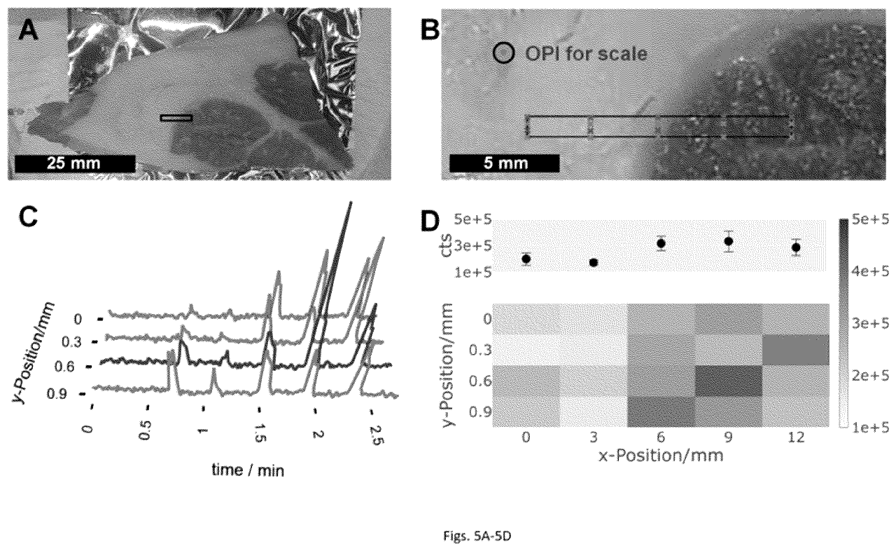

FIG. 5A shows an image of the sample. The rectangle shows the area sampled. FIG. 5B shows a closeup of the area sampled and the position of each sampling location. Across the x-axis and y-axis, the sampling locations were spaced apart 3.0 and 0.3 mm respectively. Total ion chromatograms (TIC) of each line that was sampled are shown in FIG. 5C. The probe was in contact with each location for approximately 8 s (sampling time). To quantify the amount of heme for each sampling location the TIC was smoothed to determine the center of the peak and then the XIC for (m/z=616.1±0.5) was integrated over an 8 s window. The resulting intensities are plotted in a heatmap in FIG. 5D, and the graph at the top shows average values and standard deviations of each row. As expected, the heatmap shows that that the red tissue has higher intensities of heme than the area of fatty tissue.

These results demonstrate a significant reduction in the effort required for LMJ-SSP-MS imaging across multiple locations of a biological sample. It is expected that automation of LMJ-SSP movement according to embodiments described herein will eliminate inaccuracies due to human error, provide reproducible conditions for sampling, and increase sampling throughput.

Analysis of Bacterial Colonies. An automated feedback system based on conductance measurements as described above was used in variable z height mode to sample bacterial colonies grown on agar plates. The moisture of the agar plate provided sufficient conductivity to trigger the feedback loop once the LMJ-SSP was in contact with the sample.

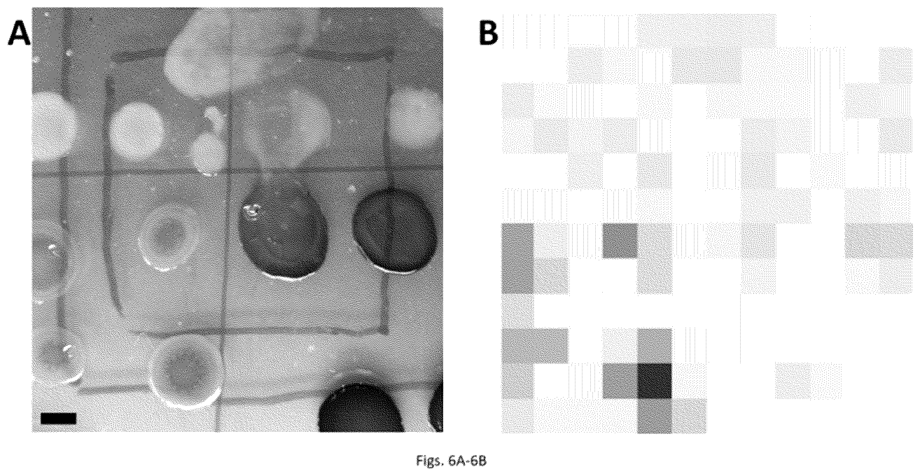

FIG. 6A shows the bacterial colonies which grew to a height above the agar surface which could lead to clogging and/or improper contact of the LMJ-SSP if the agar surface was scanned at a constant z height. At the bottom left are colonies of Pseudoalteromonas rubra, bottom right Pseudoalteromonas tunicata, top left Pseudoalteromonas elyakovii, and top right Pseudoalteromonas piscicida. The area shown in FIG. 6A was sampled at 144 equally spaced locations arranged in a 12×12 grid. FIG. 6B shows a heatmap that illustrates the spatial distribution of prodigiosin (M=323.4 g/mol, m/z=324.4±0.5), which is produced by Pseudoalteromonas rubra. As expected, prodigiosin was only detected in the sampling locations of Pseudoalteromonas rubra

These results demonstrate a significant reduction in the effort required for LMJ-SSP-MS imaging across multiple locations of biological samples. It is expected that automation of LMJ-SSP movement according to embodiments described herein will eliminate inaccuracies due to human error, provide reproducible conditions for sampling, and increase sampling throughput.

### Example 2

In one embodiment the z-axis sensor may be a conductance sensor, an example of which is shown in FIG. 2B. In this embodiment the microcontroller 208 was implemented with an Arduino® Nano™. Sensitivity of the conductance sensor may be adjusted by adjusting the value of resistor R2. Resistor R1 and the light emitting diode LED1 are optional and are used to provide a visual indication that the conductance feedback signal has been triggered when the LED1 is illuminated.

The sensor generates a feedback signal based on the voltage measured at the sample. According to this embodiment a voltage of 5V is applied to the sample through an electrically conductive sample bed (e.g., tin foil or ITO coated glass slide) and is measured via an analog input pin of the microcontroller. An analog-to-digital converter (ADC) within the microcontroller converts the voltage to a digital 10-bit value (sensorValue) between 0 (0V) and 1023 (5V), which is sent to software that controls the movement of the LMJ-SSP (see, e.g., Example 3). Once the LMJ-SSP, which has a grounded body, comes into contact with the sample, the voltage applied to the sample is drawn to ground, leading to a drop in the measured voltage which triggers the feedback signal. Therefore, a conductance value of 0 indicates no contact between the probe and sample. When there is contact between the probe and the sample the conductance value is greater than 0.

### Example 3

This example describes features of another embodiment of software for controlling automated sampling, referred to herein as “OPI Scan”.

Software was written in Python 3.1 and PyQtDesigner to control the movement of the LMJ-SSP relative to the sample by sending G-code commands via a serial USB connection to the modified 3D printer (see Example 1, FIG. 2A). The software may include a graphical user interface (GUI) such as that shown in FIG. 2C, which includes features that prompt the user for inputs. The software generates G-code commands based on the inputs, such as those shown in FIG. 2D, to sequentially move the probe towards sampling locations or “spots” that are arranged in a rectangular (or other) pattern across the sample surface. Once a sampling spot is reached, the probe resides in this position for the sampling time (“sampling time”). Following that, the probe is raised above the sampling spot where it resides for the “dwell time”, after which the probe moves above the next sampling spot. The probe then approaches this sampling spot by moving the printer head towards the sample in z-direction until solvent contact is achieved. The procedure is repeated for the remaining sampling spots. The software may allow sampling spots in “set sampling mode” or in “conductance sampling mode” using, e.g., the conductance sensor of Example 2. In set sampling mode, the probe is moved to the same z-height for each sampling spot, used for consistent height, flat sample surfaces such as sessile droplet arrays on surface energy traps or tissue samples with an even surface that is level. The conductance sampling mode is used for automated sampling of uneven and/or not leveled samples. In this mode, the probe moves stepwise towards the sample (“step distance”, e.g., 50 μm). After each step, a feedback signal from the z-axis distance sensor (i.e., the conductance sensor) is used to check for contact between the probe and the sample. Upon receiving a feedback signal from the conductance sensor indicating that the sampling spot is reached, movement of the probe in the z-direction towards the sample is stopped. The software plots the relative conductance (0 when there is no contact between the probe and sample, and >0 when there is contact between the probe and sample) and peaks are plotted. An array of relative conductance values with respective times and relative position of the probe in x, y, and z-direction is recorded and may be exported in a suitable format such as a *.csv file. The *.csv file may be used for further data analysis steps to generate height profiles and for peak alignment. A threshold value relative conductance may be defined above which the feedback signal is triggered (FIG. 2D, “threshold”). The threshold value can be adjusted depending on the electrical conductivity of the sample and the desorption solvent employed.

The software may be used in an automated feedback system based on conductance measurements as described above in variable z-axis mode (i.e., conductance mode) to create a heatmap of analyte distribution across an uneven sample surface. The measured conductance value and the z-height may be recorded at each sampling spot together with the corresponding mass spectra data. Therefore, mass spectra can be precisely assigned to their respective spatial locations even when the amount of detected analyte for a sampling spot is low. Embodiments may provided for rapid automated sampling of uneven surfaces, e.g., faster than 1 sampling spot per minute.

### Example 4

This example describes details relating to approaches for data analysis using computer software.

The software may allow mass spectra data to be acquired (e.g., in *.wiff format through Analyst (1.6.3 SCIEX, Concord, Canada)). The software may provide data analysis by first converting the *.wiff files to a suitable format such as *.mzml, e.g., using ProteoWizard (Kessner et al., Open Source Software for Rapid Proteomics Tools Development, Bioinformatics 2008, 24 (21): 2534-2536) followed by processing in a Python script using the pymzml module. The script may also import *.csv files generated by the OPI Scan software that contain recorded conductance values and positional data with its respective timestamps during each run (see Examples 1 and 3). Imported *.mzml files contain all the spectra acquired throughout each run. These data may be used by the software for various analyses. However, some spectra may be based on the composition of desorption solvent that was not in contact with the sample. Therefore, only the spectra that show the composition of desorption solvent that was in contact with the sample are of interest and need to be extracted and assigned to their respective spatial location. Different approaches may be used to identify spectra of interest. One embodiment uses the intensities of the recorded mass spectra themselves. The total ion current (TIC) of each spectrum is plotted against its respective time. Spectra of interest appear as peaks in the TIC when analytes are picked up/extracted and detected during sampling. Usually even the lowest peaks can be distinguished from the baseline (e.g., by peak picking). In another embodiment, an m/z value detected across all sampling spots is used to find the times corresponding to spectra of interest (e.g., by the extracted ion current (XIC)). However, this approach falls short for experiments where the amount of extracted and detected ions on sampling spots is low, e.g., due to low analyte concentration or a narrow measured m/z range. For reliable identification of spectra of interest independent from the recorded mass spectra, the measured conductance values may be used. According to this embodiment, recorded conductance values precisely trace the times at which the probe contacts the surface. From these contact times, the times at which mass spectra of interest appear in the MS chronogram may be extrapolated using the delay between sample contact and mass detection (t_{offset}). All spectra recorded in a defined time window around that peak time are extracted (e.g., ±4 s). The length of this time window can be adjusted based on factors such as the sampling time and the amount of analyte extracted and detected. All spectra of the same time window are averaged, leading to one mass spectrum for each sampling spot (pixel). By extracting a given m/z range from each averaged spectrum (XIC) and plotting them according to the location of their sampling spot, heatmaps that show the spatial distribution of that m/z range across the sample surface may be created. Furthermore, a surface profile can be generated when the height of the probe (z position) during sample contact (peak of conductance trace) is assigned to each pixel.

The contents of all cited publications are incorporated herein by reference in their entirety.

### EQUIVALENTS

It will be appreciated that modifications may be made to the embodiments described herein without departing from the scope of the invention. Accordingly, the invention should not be limited by the specific embodiments set forth, but should be given the broadest interpretation consistent with the teachings of the description as a whole.

1. Apparatus for automating movement of a mass spectrometry (MS) probe, comprising:
a probe holder that is adapted to hold the MS probe and is moveable relative to a sample material surface along substantially planar x, y axes and along a z axis that is substantially orthogonal to the x, y axes;
at least one z-axis sensor that outputs at least one sensor signal corresponding to a sensed z-axis position of a tip of the MS probe relative to the sample material surface; and
a controller that receives commands directing movement of the probe holder along at least the z axis, and receives the at least one sensor signal;
wherein the controller uses the at least one sensor signal in a feedback loop to set the z axis position of the MS probe tip relative to the sample material surface.

2. The apparatus of claim 1, wherein the at least one sensor signal corresponds to an electrical parameter related to a z axis position of the MS probe tip relative to the sample material surface.

3. The apparatus of claim 2, wherein the electrical parameter is conductance.

4. The apparatus of claim 3, wherein the conductance value is greater than zero when the MS probe tip is in fluid communication with the sample material surface.

5. The apparatus of claim 2, wherein the controller sets the z axis position of the MS probe tip according to a threshold value of the electrical parameter.

6. The apparatus of claim 1, comprising at least two z-axis sensors;
wherein at least a first z-axis sensor outputs a first sensor signal corresponding to a z-axis distance of the MS probe tip above the sample material surface;
wherein at least a second z-axis sensor outputs a second sensor signal corresponding to an electrical parameter related to a z-axis position of the MS probe tip upon fluid communication of the probe tip with the sample material surface.

7. The apparatus of claim 1, wherein the controller receives commands directing movement of the probe holder along the x, y, and z axes, and receives the at least one sensor signal;
wherein the controller controls movement of the MS probe to selected locations of the sample material according to the x and y axes commands, and the uses the at least one sensor signal in the feedback loop to set the z axis position of the probe tip relative to the sample material surface at each selected location.

8. The apparatus of claim 7, wherein the sample material surface is uneven and the controller uses the at least one sensor signal in the feedback loop to set different z axis positions of the probe tip relative to the sample material surface at selected locations.

9. The apparatus of claim 1, wherein the sample material comprises biological material.

10. The apparatus of claim 1, wherein the sample material comprises non-biological material.

11. The apparatus of claim 1, wherein the sample material surface is electrically conductive.

12. The apparatus of claim 1, wherein the sample material surface is not electrically conductive.

13. The apparatus of claim 1, wherein the MS probe is a liquid microjunction-surface sampling probe (LMJ-SSP).

14. A method for automating movement of an MS probe, comprising:
using a probe holder to hold the MS probe, the probe holder being moveable relative to a sample material surface along substantially planar x, y axes and along a z axis that is substantially orthogonal to the x, y axes;
providing at least one z-axis sensor that outputs at least one sensor signal corresponding to a sensed z-axis position of a tip of the MS probe relative to the sample material surface, and;
providing a controller that receives commands directing movement of the probe holder along at least the z axis, and receives the at least one sensor signal;
wherein the controller uses the at least one sensor signal in a feedback loop to set the z axis position of the MS probe tip relative to the sample material surface.

15. The method of claim 14, wherein the at least one sensor signal corresponds to an electrical parameter related to a z axis position of the MS probe tip relative to the sample material surface.

16. The method of claim 15, wherein the electrical parameter is conductance.

17. The method of claim 16, wherein the conductance value is greater than zero when the MS probe tip is in fluid communication with the sample material surface.

18. The method of claim 15, wherein the controller sets the z axis position of the MS probe tip according to a threshold value of the electrical parameter.

19. The method of claim 14, comprising providing at least two z-axis sensors;
wherein at least a first z-axis sensor outputs a first sensor signal corresponding to a z-axis distance of the MS probe tip above the sample material surface;
wherein at least a second z-axis sensor outputs a second sensor signal corresponding to an electrical parameter related to a z-axis position of the MS probe tip upon fluid communication of the probe tip with the sample material surface.

20. The method of claim 14, wherein the controller receives commands directing movement of the probe holder along the x, y, and z axes, and receives the at least one sensor signal;
wherein the controller controls movement of the MS probe to selected locations of the sample material according to the x and y axes commands, and the uses the at least one sensor signal in the feedback loop to set the z axis position of the probe tip relative to the sample material surface at each selected location.

21. The method of claim 20, wherein the sample material surface is uneven and the controller uses the at least one sensor signal in the feedback loop to set different z axis positions of the probe tip relative to the sample material surface at selected locations.

22. The method of claim 14, wherein the sample material comprises biological material.

23. The method of claim 14, wherein the sample material comprises non-biological material.

24. The method of claim 14, wherein the sample material surface is electrically conductive.

25. The method of claim 14, wherein the sample material surface is not electrically conductive.

26. The method of claim 14, wherein the MS probe is a liquid microjunction-surface sampling probe (LMJ-SSP).

27. Non-transitory computer readable media for use with a processor, the computer readable media having stored thereon instructions that direct the processor to control an apparatus for automated mass spectrometry (MS) sampling, comprising:
receiving input for selected parameters for controlling movement of an MS probe;
generating and sending commands to the apparatus to control movement of the MS probe relative to a plurality of sampling locations across a sample surface;
at each sampling location, moving the MS probe toward the sample surface in a z-direction until contact with the sample surface is achieved;
recording z-height of the MS probe at each sampling location together with corresponding MS data; and
generating an output comprising detected level of at least one analyte in the MS data at each sample location.

28. The non-transitory computer readable media of claim 27, wherein the MS probe comprises a liquid microjunction-surface sampling probe (LMJ-SSP); further comprising:
at each sampling location, moving the LMJ-SSP toward the sample surface in a z-direction until solvent contact with the sample surface is achieved.

29. The non-transitory computer readable media of claim 28, further comprising:
at each sample location, moving the LMJ-SSP toward the sample in a z-direction until a feedback signal from a z-axis distance sensor is received;
wherein the feedback indicates that solvent contact of the LMJ-SSP with the sample surface is achieved;
wherein movement of the LMJ-SSP in the z-direction towards the sample is stopped.

30. The non-transitory computer readable media of claim 29, wherein the z-axis distance sensor comprises a conductance sensor and the feedback signal indicating that solvent contact of the LMJ-SSP with the sample surface is achieved comprises a conductance value >0.

31. The non-transitory computer readable media of claim 30, wherein the software records conductance values with respective times and relative position of the LMJ-SSP in x, y, and z-direction at each sampling location; and
outputs mass spectra data assigned to respective spatial locations on the sample.
